(19) United States
(12) Patent Application Publication
(10) Pub. No.: US 2018/0264084 A1
(43) Pub. Date: Sep. 20, 2018

(54) METHODS FOR AUTOLOGOUS STEM CELL TRANSPLANTATION

(71) Applicant: Mayo Foundation for Medical Education and Research, Rochester, MN (US)

(72) Inventors: Svetomir N. Markovic, Rochester, MN (US); Luis F. Porrata, Rochester, MN (US)

(73) Assignee: Mayo Foundation for Medical Education and Research, Rochester, MN (US)

(21) Appl. No.: 15/762,213

(22) PCT Filed: Sep. 23, 2016
(86) PCT No.: PCT/US2016/053262

§ 371 Date: Mar. 22, 2018

Related U.S. Application Data
(60) Provisional application No. 62/232,253, filed on Sep. 24, 2015.

(51) Int. Cl. A61K 38/20; A61K 35/17; G01N 33/50; C12N 5/0783; A61K 45/06

(52) U.S. Cl. CPC A61K 38/2013; A61K 35/17; G01N 33/5091; C12N 5/0638; G01N 2800/52; C12N 2501/2302; C12N 2501/22; C12N 2501/24; C12N 2501/26; A61K 45/06

(57) ABSTRACT

Materials and methods for obtaining populations of lymphocytes and administering the population of lymphocytes to a subject are disclosed herein. In particular, disclosed herein are materials and methods for predicting and improving survival based on the absolute number of natural killer cells observed in the subject after transplant.

### CROSS-REFERENCE TO RELATED APPLICATIONS

This application claims benefit of priority from U.S. Provisional Application Ser. No. 62/232,253, filed on Sep. 24, 2015.

### TECHNICAL FIELD

This document relates to methods and materials for transplantation of autologous lymphocytes, and for predicting and improving survival of transplant patients.

### BACKGROUND

Autologous stem cell transplantation (ASCT) following chemotherapy can improve survival in both previously untreated multiple myeloma and relapsed, chemotherapy-sensitive, aggressive non-Hodgkin's lymphoma patients. In addition, allogeneic stem cell transplantation following chemotherapy can reduce relapse rates, which have been correlated to early absolute lymphocyte count (ALC) recovery as a manifestation of early graft-versus-tumor effect in the recipient (Kersey et al. (1987) New Engl J Med 317:416; Marmont et al. (1991) Blood 78:2120). Post-allogeneic bone marrow transplant studies have demonstrated that early ALC recovery is associated with prolonged survival (Prowles et al. (1998) Blood 91: 3481). However, allogeneic stem cell transplantation also has been associated with a higher incidence of graft-versus-host disease (GVHD).

### SUMMARY

This document provides materials and methods that combine the benefits of ASCT with the benefits of allogeneic stem cell transplantation. The disclosure herein is based on the discovery that the absolute number of natural killer cells (ANK) present in a blood sample taken from a cancer patient after ASCT can be a powerful indicator of prognosis. For example, this disclosure is based in part on the discovery that an ANK of at least 250 natural killer (NK) cells per microliter of blood post-ASCT is strongly correlated with survival. Thus, this document provides materials and methods for treating a mammalian subject (e.g., a human patient) diagnosed with cancer (e.g., breast cancer, non-Hodgkin's lymphoma, multiple myeloma, Hodgkin's disease, or acute myeloid leukemia) by ASCT, ascertaining prognosis and predicting the subject's likelihood of survival, and if the subject is determined to have a reduced likelihood of survival, treating the subject after ASCT to increase the subject's chance of survival. The methods provided herein also can be used to achieve and/or maintain an ANK of at least 250 cells per μl in an ASCT patient.

In one aspect, this document features a method for treating a patient previously administered a population of autologous lymphocytes. The method can include (a) measuring the absolute natural killer (NK) cell count (ANK) in a blood sample from the patient, and (b) when the ANK of the blood sample is less than 250 cells/μl, administering to the patient one or more agents that stimulate the function or activity of NK cells. The one or more agents can be selected from the group consisting of IL-2, IL-12, IL-15, IL-17, IL-21, IFN-alpha, and IFN-gamma. For example, the agent can be IL-2. The patient can be a patient diagnosed with cancer, such as breast cancer, non-Hodgkin's lymphoma, multiple myeloma, Hodgkin's disease, or acute myeloid leukemia. Prior to collection of the autologous lymphocytes from the patient, or prior to administration of the autologous lymphocytes to the patient, the patient can be in remission from the cancer. The method can further include administering the population of autologous lymphocytes to the patient. The method can further include collecting the population of autologous lymphocytes from the patient. The collecting can include (i) collecting from the patient a biological sample comprising NK cells, (ii) monitoring the number of collected NK cells in the sample, and (iii) repeating steps (i) and (ii) until the total number of collected NK cells is at least 0.09×10⁹ cells per kg. The method can further include, prior to collecting the biological sample, administering to the patient one or more agents that stimulate NK cell function or activity (e.g., one or more agents selected from the group consisting of IL-2, IL-12, IL-15, IL-17, IL-21, IFN-alpha, and IFN-gamma). The method can further include, prior to collecting the biological sample, administering to the patient one or more agents that can (i) stimulate proliferation of stem cells and/or progenitor cells, and/or (ii) stimulate mobilization of stem cells and/or progenitor cells to the peripheral circulation. The one or more agents can be selected from the group consisting of G-CSF, GM-CSF, SCF, IL-2, IL-7, IL-8, IL-12, and flt-3 ligand. The method can further include, prior to administering the autologous lymphocytes to the patient, contacting the autologous lymphocytes with one or more agents that stimulate function or activity of NK cells (e.g., one or more agents selected from the group consisting of IL-2, IL-12, IL-15, IL-17, IL-21, IFN-alpha, and IFN-gamma). The method can further include, prior to administering the autologous lymphocytes to the patient, subjecting the patient to an immunosuppressive treatment. The immunosuppressive treatment can be radiotherapy or chemotherapy, or surgery with anesthesia.

In another aspect, this document features a method for identifying a cancer patient treated by autologous stem cell transplant (ASCT) as having an increased likelihood of survival. The method can include (a) measuring the ANK in a blood sample from the patient and (b) when the ANK in the blood sample is at least 250 cells/μl, identifying the patient as having an increased likelihood of survival. The method can include measuring the ANK three months after ASCT, six months after ASCT, nine months after ASCT, or twelve months after ASCT.

In addition, this document features a method for identifying a cancer patient treated by ASCT as having a decreased likelihood of survival. The method can include (a) measuring the ANK in a blood sample from the patient, and (b) when the ANK in the blood sample is less than 250 cells/μl, identifying the patient as having a decreased likelihood of survival. The method can include measuring the ANK three months after ASCT, six months after ASCT, nine months after ASCT, or twelve months after ASCT.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and example are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1D are a series of graphs plotting survival of non-Hodgkins lymphoma patients after ASCT, based on the absolute numbers of lymphocytes or NK cells in the transplanted cells. FIG. 1A, overall survival for patients who received ASCT with an ALC of at least 0.5×10⁹ lymphocytes/kg vs. patients who received transplants with an ALC less than 0.5×10⁹ lymphocytes/kg. FIG. 1B, progression-free survival for patients who received ASCT with an ALC of at least 0.5×10⁹ lymphocytes/kg vs. patients who received transplants with an ALC less than 0.5×10⁹ lymphocytes/kg. FIG. 1C, overall survival for patients who received ASCT with an autologous NK cell count (A-NK) of at least 0.09×10⁹ cells/kg vs. patients who received transplants with an A-NK less than 0.09×10⁹ cells/kg. FIG. 1D, progression-free survival for patients who received ASCT with an A-NK of at least 0.09×10⁹ cells/kg vs. patients who received transplants with an A-NK less than 0.09×10⁹ cells/kg. Patients who received ASCT with an ALC of at least 0.5×10⁹ lymphocytes/kg had higher rates of overall (P<0.01) and progression-free (P<0.002) survival than patients who received ASCT with an ALC less than 0.5×10⁹ lymphocytes/kg. Patients who received ASCT with an A-NK of at least 0.09×10⁹ cells/kg had a much higher rate of overall (P<0.004) and progression-free (P<0.0001) survival than patients who received ASCT with an A-NK less than 0.09×10⁹ cells/kg. The median follow-up was 57.2 months.

FIGS. 2A-2D are a series of graphs plotting overall survival of non-Hodgkins lymphoma patients who were treated by ASCT and then monitored for ANK counts at three months (FIG. 2A), six months (FIG. 2B), nine months (FIG. 2C), and 12 months (FIG. 2D) after transplant. At all time points of ANK determination, patients with an ANK greater than or equal to 250 cells/μl had a vastly improved chance of overall survival to at least 84 months post-transplant, with nearly 100% survival rates (P<0.0001 for all time points). The median follow-up was 57.2 months.

FIGS. 3A-3D are a series of graphs plotting progression-free survival of non-Hodgkins lymphoma patients who were treated by ASCT and then monitored for ANK counts at three months (FIG. 3A), six months (FIG. 3B), nine months (FIG. 3C), and 12 months (FIG. 3D) after transplant. At all time points of ANK determination, patients with an ANK greater than or equal to 250 cells/μl had a vastly improved chance of progression-free survival to at least 84 months post-transplant (P<0.0001 for all time points). The median follow-up was 57.2 months.

### DETAILED DESCRIPTION

This document provides materials and methods for using ANK to predict cancer patient prognosis, based at least in part on the discovery that the number of NK cells within a population of blood cells in an ASCT recipient can be correlated with survival. This document also provides materials and methods for treating an ASCT recipient to achieve an ANK of at least 250 cells/μl of blood.

As used herein, “autologous” as it relates to transplantation refers to a graft in which the donor and recipient is the same individual. Thus, in an autologous transplant cells are harvested from a subject and then returned to the same subject. In contrast, an “allogeneic” transplant refers to a graft in which the donor and recipient are genetically non-identical individuals from the same species. A “xenogeneic” transplant refers to a graft in which the donor and recipient are of different species.

As used herein, an ASCT refers to a procedure in which a sample of a subject's own stem cells are removed and subsequently transplanted back into the same subject. Stem cells can be harvested from bone marrow or peripheral blood, for example. Once obtained, stem cells can be frozen until needed. For example, stem cells can be obtained from a patient, cryopreserved (e.g., at a temperature≤−85° C.), and then thawed and returned (i.e., transplanted, typically by transfusion) to the patient. In some embodiments, stem cell aliquots can be thawed, loaded into one or more sterile syringes or infusion bags, and injected intravenously over a period of time.

In some embodiments, stem cells capable of reconstituting a patient's immune system can be obtained from the patient's peripheral circulation following mobilization of such cells from bone marrow into peripheral blood. Mobilization of stem cells can be accomplished by treatment of a patient with one or more factors that can (i) stimulate an increase in proliferation of stem cells and/or progenitor cells, and/or (ii) stimulate migration of stem cells and/or progenitor cells from the bone marrow into the peripheral circulation. Such factors can be administered with adjuvants and/or other accessory substances, separately or in combination as desired. Examples of factors that can be used in this aspect include, without limitation, granulocyte colony-stimulating factor (G-CSF), granulocyte/macrophage colony-stimulating factor (GM-CSF), c-kit ligand (stem cell factor (SCF)), interleukin-2, -7, -8, and -12 (IL-2, IL-7, IL-8, and IL-12), and flt-3 ligand. See, e.g., Bungart et al. (1990) Br. J. Haematol. 76:174; Terella et al. (1993) Bone Marrow Transplant. 11:271; Molineux et al. (1991) Blood 85:275; Grzegorzewski et al. (1994) Blood 83:377; Laterveer et al. (1995) Blood 85:2269; Jackson et al. (1995) Blood 85:2371; and Lyman et al. (1994) Blood 83:2795. Factors to be administered can include, for example, G-CSF alone (e.g., 10 μg/kg/day G-CSF), G-CSF+flt-3 ligand (e.g., 10 μg/kg/day G-CSF+50 μg/kg/day flt-3 ligand), or GM-CSF+flt-3 ligand (e.g., 5 μg/kg/day GM-CSF+50 μg/kg/day flt-3 ligand). See, e.g., Sudo et al. (1997) Blood 89:3186. Such factors can be administered prior to harvest or starting on the day of harvest, for example, and can be given on a daily basis for one to seven days (e.g., for one, two, three, four, five, six, or seven days), or until stem cell harvesting is complete. Factors that stimulate stem cell proliferation or mobilization can be administered using any suitable method. Typically, such factors can be administered parenterally (e.g., by subcutaneous, intrathecal, intraventricular, intramuscular, or intraperitoneal injection, or by intravenous drip). Mobilization of stem cells with, for example, GM-CSF and flt-3 ligand can be evaluated by determining the number of CD34⁺ cells present before, during, and/or after treatment with one or more mobilizing agents. In some embodiments, the number of CD34⁺ cells can be determined by FACS analysis using CD34-specific antibodies conjugated to fluorescent or other labeling moieties.

Following or during mobilization, peripheral blood stem cells (PBSC) can be collected using, for example, an apheresis procedure. The process of apheresis, which is well known in the art, involves removal of whole blood from a patient or donor. Within an instrument that is essentially designed as a centrifuge, the components of the whole blood are separated. One or more of the separated portions is then withdrawn, and the remaining components can be retransfused into the patient or donor. Thus, for example, all or most (e.g., 80%, 90%, 95%, 99%, or 100%) of the erythrocytes in a sample of whole blood can be returned to a patient during an apheresis procedure, while lymphocytes (e.g., NK cells) and stem cells can be collected. Apheresis can be performed as many as four times per week (e.g., one, two, three, or four times per week). In some embodiments, a commercially available blood cell collection device can be used, such as the CS3000® blood cell collection device marketed by the Fenwal Division of Baxter Healthcare Corporation (Fenwal Laboratories, Deerfield, Ill.). Methods for performing apheresis with the CS3000® machine are described in Williams et al. (1990) Bone Marrow Transplantation 5:129-33, and Hillyer et al. (1993) Transfusion 33:316-21, for example, both of which are incorporated herein by reference in their entirety.

Typically, a total blood volume between 9.5 and 10 L per apheresis procedure can be processed at a flow rate of 50 to 70 ml/min. Following collection, a cell count can be performed on an aliquot of the total product to determine the number of stem cells. Cells can be collected until the total sample taken from the patient reaches a concentration of at least 1×10⁶ CD34⁺ stem cells/kg (e.g., at least 2×10⁶ CD34+ cell/kg, or at least 3×10⁶ CD34+ cells/kg).

Despite various methods of PBSC mobilization, adequate numbers of PBSC for ASCT may be not collected from some patients during a single apheresis procedure. In these patients, bone marrow harvest or a second attempt at PBSC mobilization can be performed. Alternatively, these patients may be excluded from proceeding to ASCT.

Apheresis products can be centrifuged, and the plasma can be removed to yield a total volume of, for example, about 100 ml. The resulting cell suspension can be mixed with a physiological freezing solution [e.g., minimal essential medium such as MEM-S (Invitrogen Life Technologies, Carlsbad, Calif.) supplemented with 20% dimethylsulfoxide (DMSO)]. Cell/media suspensions can be transferred to freezing bags or any other freezing receptacle known in the art, and frozen to −100° C. using, for example, a computer-controlled cryopreservation device (e.g., the Cryoson-BV-6; Cryoson Deutschland GmbH, FRG). The cells then can be transferred into liquid nitrogen and stored until transplantation.

Patients typically undergo a pre-transplant workup to evaluate, for example, heart, liver, kidney, and lung function, as well as current disease status. In some embodiments, patients deemed to be eligible (e.g., healthy enough) for ASCT are subjected to a tumor debulking procedure prior to ASCT. For example, a patient can be treated with high doses of chemotherapy, radiation therapy, and/or surgery (e.g., surgery with anesthesia) before the transplant. Stem cells for transplant typically are collected prior to tumor debulking regimens, since such potentially lethal procedures can be immunosuppressive and can severely damage or destroy the bone marrow. ASCT following a debulking procedure can reconstitute the patient's immune cells with stem cells present in the transplant.

In some embodiments, a patient's stem cells can be collected by bone marrow harvest using procedures known in the art, or by a stem cell apheresis procedure as described above, for example. Collected stem cells can be cryopreserved, and the patient can undergo a debulking procedure such as high-dose chemotherapy and/or radiation therapy. After the debulking procedure is completed, the patient's stem cells can be transplanted. ASCT can be done almost immediately after a debulking procedure (e.g., 24 to 48 hours after high-dose chemotherapy). Alternatively, a longer period of time (e.g., a week to several months) can elapse between a debulking procedure and ASCT. Due to the likelihood of immunosuppression as a result of the debulking procedure, protective isolation precautions generally are taken after ASCT at least until the reinfused stem cells begin to engraft. “Engraftment” refers to a process whereby the transplanted stem cells begin to differentiate into mature blood cells. In addition, stem cells can be treated prior to transplantation with, for example, anticancer drugs or antibodies to reduce the number of cancerous cells that may be present in the sample. Such procedures are referred to as “purging.”

In the methods provided herein, patients can be treated by administration of autologous cell populations that can contain stem cells and other cell types, including, for example, red blood cells (RBC) and lymphocytes. Lymphocytes are white blood cells (WBC) that are formed in lymphatic tissue throughout the human body (e.g., lymph nodes, spleen, thymus, tonsils, Peyer's Patches, and bone marrow). In normal adults, lymphocytes comprise approximately 22% to 28% of the total number of leukocytes in the circulating blood. As used herein, the term “lymphocyte” includes NK cells, B cells, and T cells (e.g., T helper cells, cytotoxic T cells, and T suppressor cells). NK cells are directly cytotoxic to foreign cells (e.g., foreign cancer cells), and do not require complement activity to effect their lysis. NK cells represent the body's first line of defense against malignancy. B cells produce immunoglobulins, and T cells are involved in modulation of immune responses and in regulation of erythropoiesis. Different types of lymphocytes can be distinguished from each other and from other cell types based on the cell type-specific expression of particular molecular markers, generally cell surface markers. For example, NK cells bear the markers CD16 and/or CD56 on their surface. B cells bear at least one of the cell surface markers CD19 and CD20. T cells bear one or more of the cell surface markers CD3, CD4, and CD8. Typically, cytotoxic T cells express CD8, whereas helper T cells express CD4.

As used herein, the term “absolute lymphocyte count” (ALC) refers to the total number of lymphocytes per unit of whole blood or blood cells in a sample or in a subject (e.g., a human patient). A unit can be, for example, a liter (L), milliliter (ml), or microliter (μl). Typically, but not always, ALC is measured as the number of mature lymphocytes per microliter of blood, and includes the cumulative numbers of B cells, T cells, and NK cells. Stem cells, lymphocyte precursor cells, and lymphocyte progenitor cells typically are not included in the ALC. Stem cells can be differentiated from lymphocytes in that stem cells express the cell surface marker CD34, whereas mature lymphocytes do not. Moreover, lymphocytes express specific cell surface markers as described above, whereas stem cells do not express these markers.

To determine an ALC, a sample of blood can be collected from a patient. For example, blood can be collected in a rubber-stopped tube containing EDTA or another medically acceptable anti-coagulant. Blood can be collected using any route of entry to the circulatory system known in the art. The blood sample then can be analyzed to determine the ALC. In one embodiment, an ALC can be obtained using an automated system for counting blood cells in a sample. Such cell counting systems typically are based on a principle by which unstained, unlabeled cells are sorted and counted based on morphological characteristics including, without limitation, cell size, cell shape, nuclear size, and nuclear shape. For example, the GEN-S™ Hematology Analyzer identifies and counts cell types based on three general criteria: volume, conductivity, and scatter (see U.S. Pat. No. 5,125,737). A blood sample can be treated before analysis with reagents and/or physical agitation to lyse the RBC, thereby leaving WBC for analysis. The Gen-S™ Analyzer uses a process of direct current impedance by which the cells are collided with light to physically measure the volume displaced by the entire cell in an isotonic diluent. Cell size thus can be accurately determined regardless of the orientation of the cell in the light path. Cells can be further collided with an alternating current in the radio frequency range that can permeate cell membranes, such that information can be obtained with regard to internal structure including, for example, chemical composition and nuclear structure. A cell can be collided with a laser beam that, upon contacting the cell, scatters and spreads out in all directions, generating median angle light scatter signals. These signals can be collected to yield information regarding cellular granularity, nuclear lobularity, and cell surface structure. Thus, such a system can count and differentiate RBC from WBC based on the presence or absence of a nucleus, and can count and differentiate the different types of WBC based on the ratio of nuclear to cytoplasmic volume, lobularity of the nucleus, and granularity of the cytoplasm as described below, for example.

ALC also can be determined by placing a known volume of a blood sample onto a glass microscope slide, smearing the sample to create a thin film of blood on the slide, and staining the sample using standard histological stains such as, for example, hematoxylin and eosin (H&E). Briefly, a blood smear can be dried and subsequently fixed onto a slide using a fixative such as, without limitation, neutral buffered formalin, formaldehyde, paraformaldehyde, glutaraldehyde, Bouin's solution, mercuric chloride, or zinc formalin. The slides then can be immersed in a solution of Harris Hematoxylin, rinsed in water, immersed in a solution of Eosin, rinsed in water, dehydrated in ascending alcohol solutions, and cleared in xylenes. In blood smears that have been stained using H&E, nuclei and other basophilic structures stain blue, whereas cytoplasm and other acidophilic structures stain light to dark red (see, Sheehan et al. (1987) Theory and Practice of Histotechnology, 2nd Edition, Battelle Memorial Institute, Columbus, Ohio, which is incorporated herein by reference in its entirety). The number of lymphocytes present in a blood smear can be counted based on lymphocytic morphological criteria accepted in the art.

For example, when stained with H&E, the lymphocyte nucleus is deeply colored (purple-blue) and is composed of dense aggregates of chromatin within a sharply defined nuclear membrane. The nucleus generally is round, eccentrically located, and surrounded by a small amount of light blue staining cytoplasm. The volume of nucleus to cytoplasm in a lymphocyte typically is about 1:1.2. Lymphocytes can be differentiated from RBC in that RBC have no nuclei. Lymphocytes can be differentiated from neutrophils in that neutrophils have nuclei with 2 to 5 lobes, while lymphocyte nuclei are not lobed. Lymphocytes can be differentiated from basophils and eosinophils in that those cells have cytoplasmic granules, while lymphocytes do not have cytoplasmic granules. Lymphocytes can be differentiated from monocytes in that monocytes are 16 to 20 μm in diameter, while lymphocytes are 7 to 10 μm in diameter. In addition, one of skill in the art may refer to any of a number of hematology or histological texts or atlases (e.g., Wheater et al. (1987) Functional Histology 2nd Ed. Churchill Livingstone, incorporated herein by reference in its entirety) to determine the physical appearance of a lymphocyte.

ALC also can be determined by immunolabeling lymphocytes with antibodies specific for lymphocyte cell surface markers, and counting the immunolabeled cells using fluorescence flow cytometry (FFC). For example, NK cells can be labeled with one or more fluorescently labeled antibodies specific for CD16 and/or CD56. Similarly, B cells can be labeled with one or more fluorescently labeled antibodies specific for the adhesion molecules CD20 and/or CD19, and T cells can be labeled with one or more fluorescently labeled antibodies specific for CD3, CD4, and/or CD8, and. To determine ALC, cell surface marker-specific antibodies can be labeled with the same fluorophore (e.g., Cy-5, fluorescein, or Texas Red). In a FFC procedure, individual cells are held within a thin stream of fluid and passed through one or more laser beams, one cell at a time, causing light to scatter and the fluorescent dyes to emit light at various predetermined frequencies. Photomultiplier tubes convert the light to electrical signals, allowing for quantitation of the number of cells bearing the fluorophore. If all lymphocyte subtypes are labeled with the same fluorophore, quantification of the number of fluorophore-bearing cells will yield an ALC. FFC and quantitation is further described in, for example, U.S. Pat. No. 4,499,052. In addition, a FFC machine can be adapted for fluorescence activated cell sorting (FACS), which involves the separation (and collection) of (a) fluorescent cells from non-fluorescent cells; (b) strongly fluorescent cells from weakly fluorescent cells; or (c) cells fluorescing at one wavelength from cells fluorescing at another wavelength.

The methods provided herein are based on determining the ANK in ASCT transplant recipients, as an ANK of at least 250 cells/μl of blood has been correlated with increased survival of patients following tumor debulking and ASCT. As used herein an “ANK” refers to the absolute number of NK cells in a population of lymphocytes. In some embodiments, an ANK can be calculated by multiplying the ALC in a blood sample by the percentage of NK cells in the lymphocyte population. Samples obtained any time between 2 weeks and 15 months (e.g., one to three months, two to four months, three to six months, six to nine months, nine to 12 months, or 12 to 15 months) following completion of an ASCT can be particularly useful. Completion of an ASCT occurs at that time when all of the stem cells intended for transplant have been administered to the patient.

In the methods provided herein, patients (e.g., cancer patients who have undergone ASCT) can be treated to achieve and/or maintain an ANK of at least 250 cells/μl (e.g., at least 500 cells/μl, at least 1000 cells/μl, at least 5000 cells/μl, or at least 10,000 cells/μl). The methods can include, for example, obtaining from a patient (e.g., a human cancer patient) a population of cells containing lymphocytes, as described herein, administering the population of cells back to the patient, measuring the ANK in a blood sample from the patient at one or more time points following administration of the cells, predicting the patient's likelihood of survival based on the ANK, and when the ANK indicates a reduced chance of survival, administering to the patient an agent that can stimulate an increase in the number of NK cells within the patient (e.g., such that an ANK of at least 250 cells/μl is reached).

In some embodiments, the population of lymphocytes collected from and administered back to the patient can contain a particular number of NK cells. For example, the methods provided herein can include the following steps: (a) collecting from a patient a biological sample (e.g., a blood sample) containing NK cells, (b) monitoring the number of NK cells in the collected sample, and (c) repeating steps (a) and (b) until the total number of collected NK cells is at least about 0.05×10⁹ cells/kg weight of the patient (e.g., at least about 0.07×10⁹ cells/kg, at least about 0.08×10⁹ cells/kg, at least about 0.09×10⁹ cells/kg, at least about 0.1×10⁹ cells/kg, or at least about 0.12×10⁹ cells/kg). The patient can be a human cancer patient diagnosed with, for example, non-Hodgkin's lymphoma, Hodgkin's disease, multiple myeloma, acute myeloid leukemia, or breast cancer. NK cells can be collected from the patient using, for example, an apheresis procedure as described above, and the number of collected NK cells can be determined using, for example, immunolabeling with one or more fluorescently labeled antibodies specific for CD16 and/or CD56, and counting with FACS. Typically, the cell population can be returned to the patient by intravenous infusion, although any suitable method known in the art can be used. In some embodiments, the patient can be in remission from the cancer, either prior to collection of the cells or prior to returning the cells to the patient.

In some cases, the methods provided herein also can include treatment of a patient or a cell population (e.g., in a biological sample such as an apheresis product) with one or more agents that stimulate proliferation, maturation, differentiation, function, and/or activity of immune cells (e.g., NK cells). For example, a patient can be treated with an NK enhancing agent such as an agent such as IL-2, IL-12, IL-15, IL-17, IL-21, or an interferon (IFN; e.g., IFN-α or IFN-γ) prior to collection of a biological sample containing NK cells, in order to increase the number of NK cells in the population collected for transplant.

In some embodiments, a population of cells (e.g., a population of collected autologous lymphocytes containing NK cells) can be contacted in vitro with one or more agents such as those listed above. For example, collected cells can be placed in a vessel (e.g., a bag, a tube, a vial, or any other suitable container) and contacted with one or more agents such as those described above. In some embodiments, for example, NK cells can be contacted in vitro with IL-2 at a dose of, for example, about 1.5×10⁶ to about 2.0×10⁶ units. NK cell enhancing agents can be added to cells within a container such as a bag (e.g., a blood bag), tube, or vial, or such a vessel can contain one or more such agents prior to placement of cells within the vessel. In some embodiments, one or more agents can be dispersed on an inner surface of the vessel. For example, an agent in liquid form can be dispersed (e.g., sprayed) onto an inner surface of the vessel and allowed to dry. Alternatively, an agent in solid (e.g., lyophilized or powdered) form can be dispersed on an inner surface of the vessel, or an agent in liquid or solid form can simply be placed within the vessel. ASCT using a population of lymphocytes that has been treated with one or more NK cell enhancing agents may increase the likelihood that the ANK determined after transplant will be at or above the threshold (250 cells/μl) associated with a high likelihood of survival.

In some embodiments, such as when the ANK in an ASCT recipient is less than 250 cells/μl of blood (such that the patient is identified as having a reduced chance of survival), the patient can be treated with an agent such as IL-2, IL-12, IL-15, IL-17, IL-21, or an IFN (e.g., IFN-α or IFN-γ), in order to increase the ANK in the patient, and to increase the patient's chance of survival. These agents can be native factors obtained from a natural source, factors produced by recombinant DNA methodology, chemically synthesized polypeptides or molecules, or any derivative having the functional activity of the native factor. Agents such as those listed above can be administered to a patient via any pharmaceutically acceptable route known in the art, including, for example, intravenous injection, intra-arterial injection, subcutaneous injection, intramuscular injection, intraperitoneal injection, or oral administration in the form of a tablet, capsule, or syrup. In some embodiments, IL-2 can be administered to a patient prior to collection of NK cells or after ASCT. In other embodiments, a patient can be treated with IFN at a concentration of, for example, between about 1×10⁵ and about 1×10⁷ units/m². The agent(s) can be administered at any time from the day of transplant up to 24 months or more (e.g., one week, two weeks, one month, two months, three months, four months, six months, eight months, 10 months, 12 months, 15 months, 18 months, 24 months, or more than 24 months) following the transplant. Further, administration of the agent(s) can be repeated (e.g., daily, two to four times per week, weekly, every other week, two to eight times per month, monthly, every other month, every third month, or two to ten times per year). The treatment regimen can be determined based on ANK counts obtained on a repeated or recurring basis after ASCT. For example, if a patient demonstrates an ANK less than 250 cells/μl blood at any time after ASCT, the patient can be treated with one or more agents that can stimulate proliferation, maturation, differentiation, function, and/or activity of NK cells, and the ANK can be reassessed at a later time point. Treatment can be repeated if the ANK does not increase to at least 250 cells/μl blood within a predetermined length of time (e.g., two to four days, a week, two weeks, or a month). In some embodiments, treatment with the one or more agents can continue even after an ANK of at least 250 cells/μl of blood has been reached. Alternatively, treatment with the one or more agents can be discontinued once the ANK reaches at least 250 cells/μl blood, and can be initiated again if the ANK drops below 250 cells/μl.

Patients or biological samples containing NK cells and other lymphocytes also can be treated with one or more agents that activate the T cell signal transduction pathway, leading to lymphocyte activation. A T cell activator can be, without limitation, one or more of the following: IL-1, IL-2, IL-4, IL-5, IL-6, IL-7, IL-12, IL-13, IFNα, IFNγ, tumor necrosis factor (TNFα), an anti-CD3 antibody or antigen-binding fragments thereof (anti-CD3), an anti-CD28 antibody or antigen-binding fragments thereof (anti-CD28), phytohemagglutinin, concanavalin-A, and phorbol esters. As above, these agents can be native factors obtained from a natural source, factors produced by recombinant DNA methodology, chemically synthesized polypeptides or molecules, or any derivative having the functional activity of the native factor.

The invention will be further described in the following example, which does not limit the scope of the invention described in the claims.

### EXAMPLE

### Transplant and Post-Transplant ANK Values are Correlated with Survival

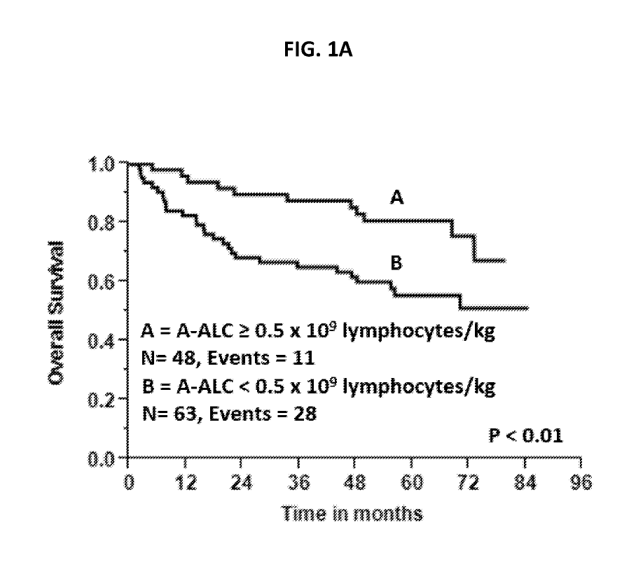

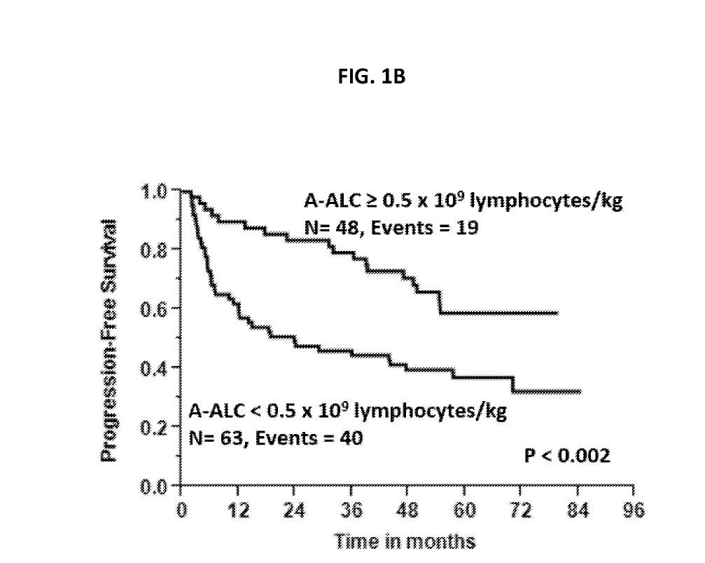

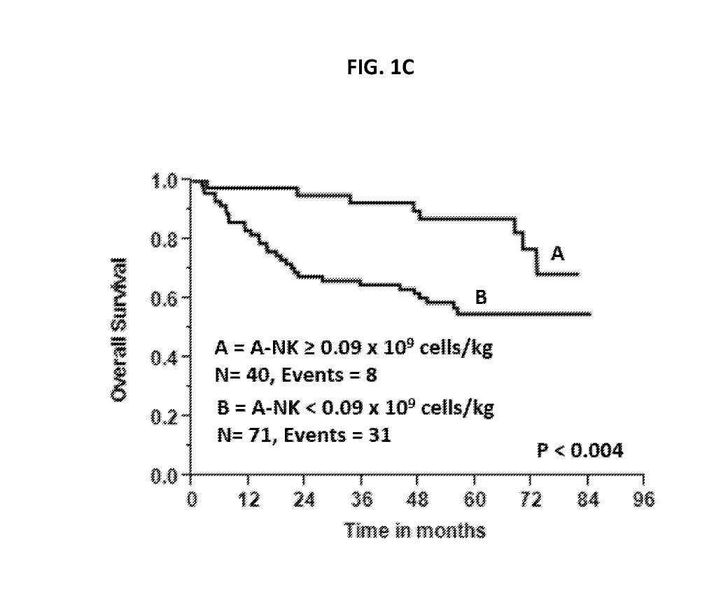

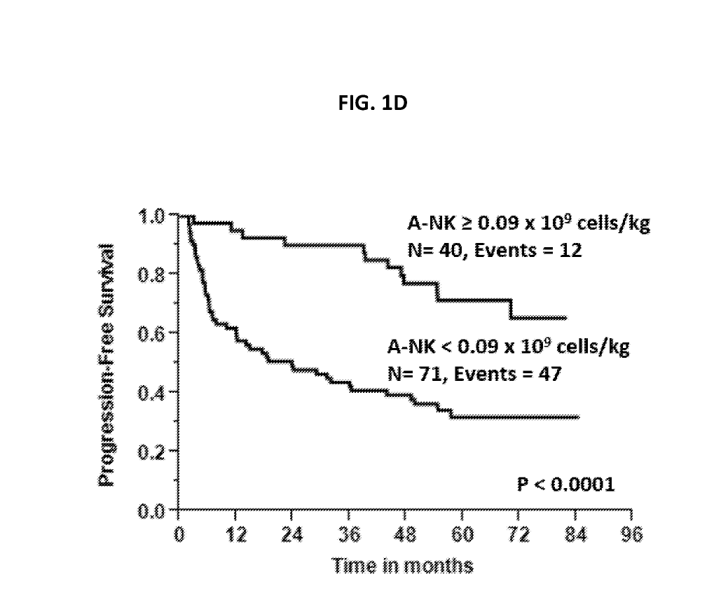

Non-Hodgkin's lymphoma patients who received autologous peripheral blood stem cell transplantation were included in these studies. Apheresis was used to collect lymphocytes containing a median of 0.06×10⁹ NK cells/kg body weight (range: 0.007-0.2) from each patient (compared with 0.05×10⁹ NK cells/kg; range: 0.005-0.2 for the standard). The lymphocytes were then infused back into the patients as autologous transplants. Patients who were infused with autologous cell populations having an ALC of at least 0.5×10⁹ lymphocytes/kg demonstrated improved overall (FIG. 1A) and progression-free (FIG. 1B) survival for up to 84 months, as compared to patients who received transplants with an ALC less than 0.5×10⁹ lymphocytes/kg. Further, patients who were infused with autologous cell populations having an A-NK of at least 0.09×10⁹ cells/kg demonstrated improved overall (FIG. 1C) and progression-free (FIG. 1D) survival for up to 84 months, as compared to patients who received transplants with an A-NK less than 0.09×10⁹ cells/kg.

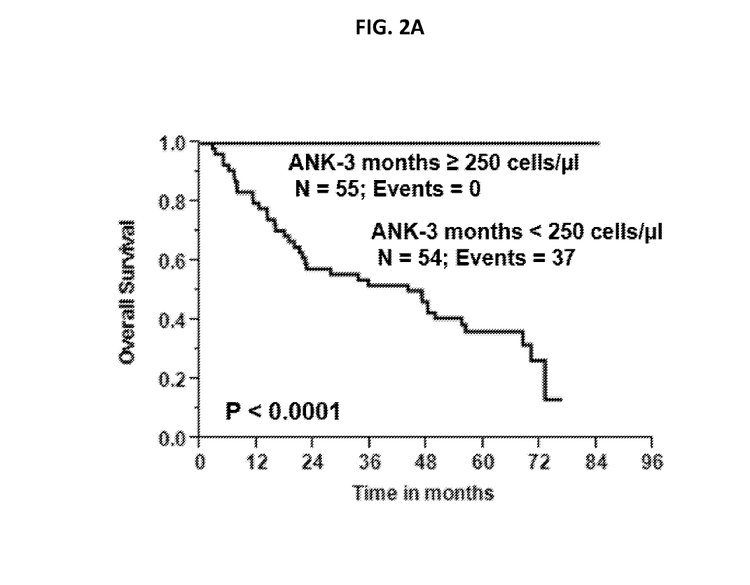

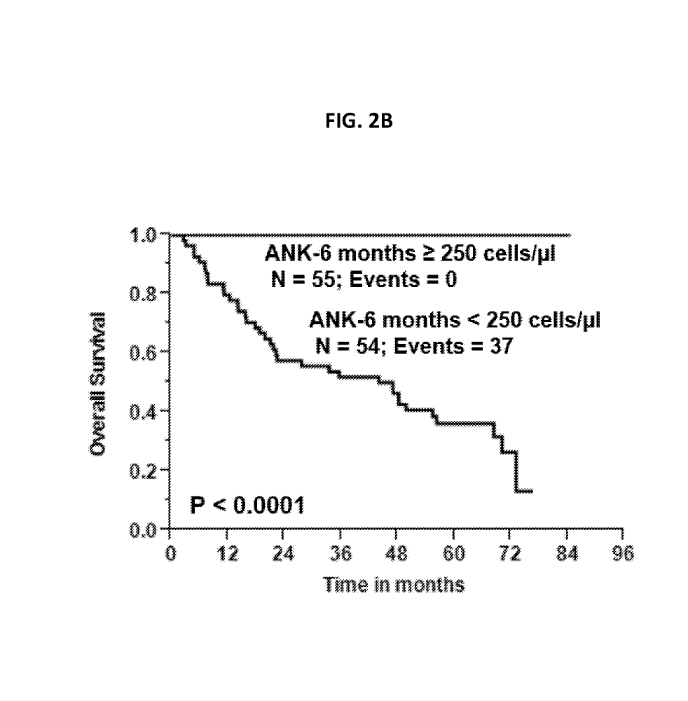

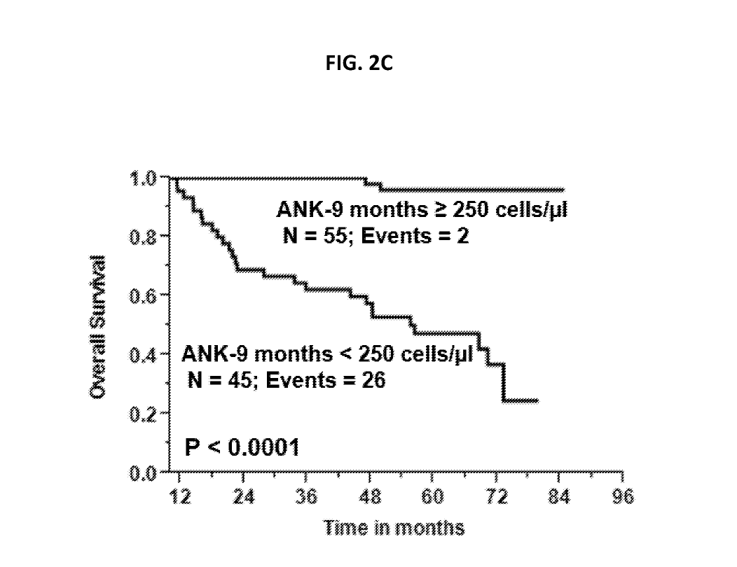

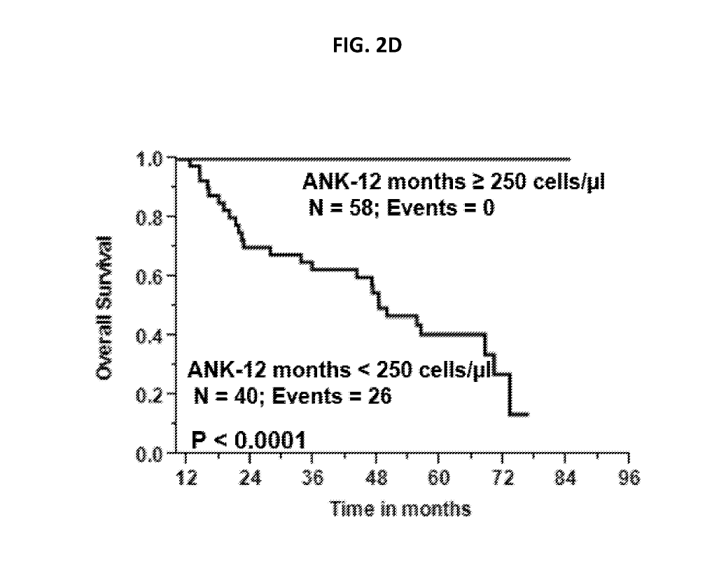

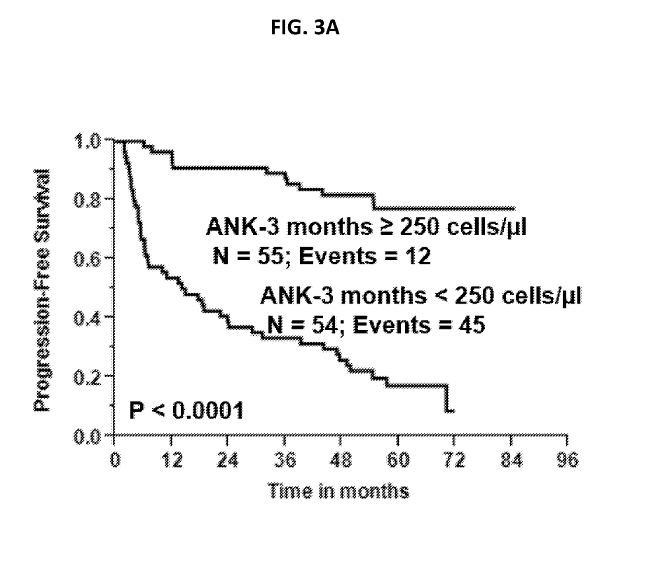

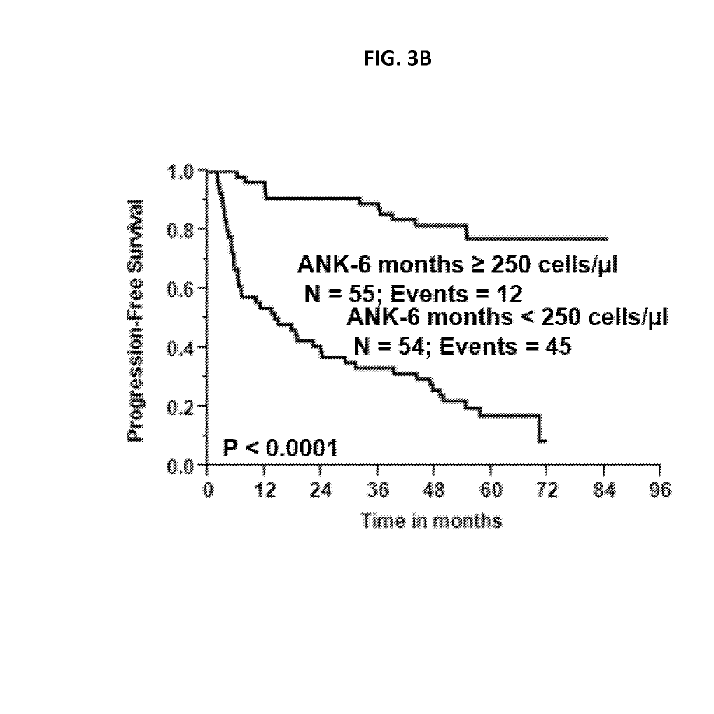

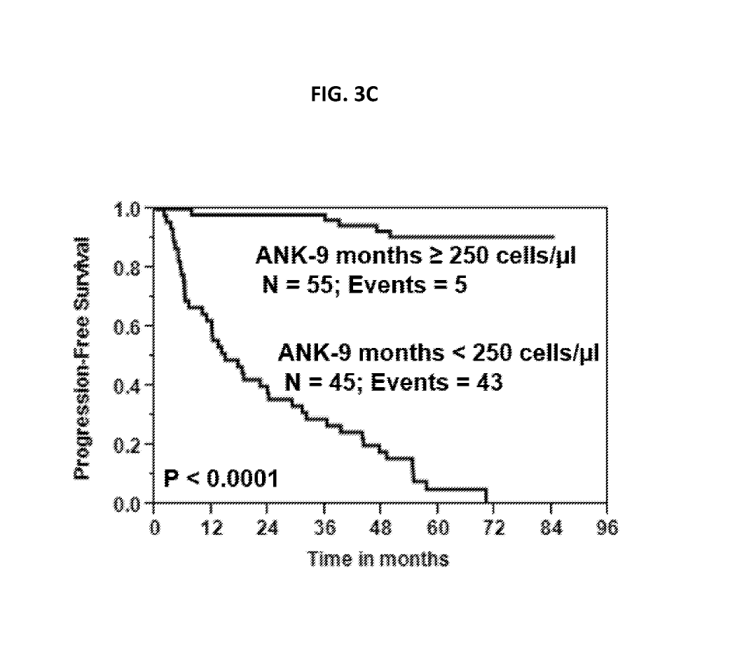

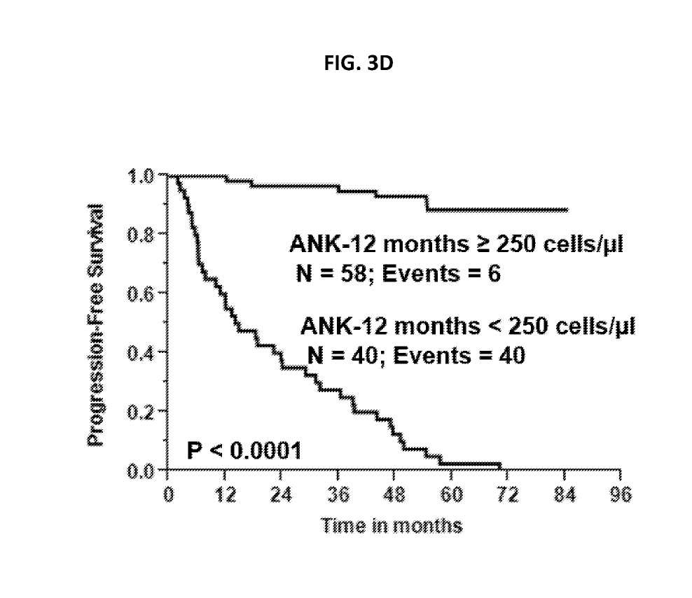

In addition, ANK counts were determined three, six, nine, and 12 months after ASCT. As shown in FIGS. 2A-2D, patients who had an ANK of at least 250 cells/μl at any of those time points had a nearly 100% chance of overall survival for at least 84 months post-transplant—significantly higher than the overall survival demonstrated by patients who had an ANK less than 250 cells/μl at three, six, nine, or 12 months after transplant (P<0.0001 at all time points). Further, as shown in FIGS. 3A-3D, patients who had an ANK of at least 250 cells/μl at any of those time points had significantly higher rate of progression-free survival for at least 84 months post-transplant than patients who had an ANK less than 250 cells/μl at three, six, nine, or 12 months after transplant (P<0.0001 at all time points). Thus, the ANK post-transplant can be indicative of a patient's prognosis, and can serve as an indicator of potential post-transplant treatment needs.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a patient previously administered a population of autologous lymphocytes, comprising:
(a) measuring the absolute natural killer (NK) cell count (ANK) in a blood sample from the patient; and
(b) when the ANK of the blood sample is less than 250 cells/μl, administering to the patient one or more agents that stimulate the function or activity of NK cells.

2. The method of claim 1, wherein the one or more agents are selected from the group consisting of IL-2, IL-12, IL-15, IL-17, IL-21, IFN-alpha, and IFN-gamma.

3. The method of claim 2, wherein the agent is IL-2.

4. The method of claim 1, wherein the patient is diagnosed with cancer.

5. The method of claim 4, wherein the cancer is breast cancer, non-Hodgkin's lymphoma, multiple myeloma, Hodgkin's disease, or acute myeloid leukemia.

6. The method of claim 5, wherein said cancer is non-Hodgkin's lymphoma.

7. The method of claim 4, wherein prior to collection of the autologous lymphocytes from the patient, the patient was in remission from the cancer.

8. The method of claim 4, wherein prior to administration of the autologous lymphocytes to the patient, the patient was in remission from the cancer.

9. The method of claim 1, further comprising administering the population of autologous lymphocytes to the patient.

10. The method of claim 9, further comprising collecting the population of autologous lymphocytes from the patient.

11. The method of claim 10, wherein the collecting comprises:
(i) collecting from the patient a biological sample comprising NK cells;
(ii) monitoring the number of collected NK cells in the sample; and
(iii) repeating steps (i) and (ii) until the total number of collected NK cells is at least 0.09×10⁹ cells per kg.

12. The method of claim 10, further comprising, prior to collecting the biological sample, administering to the patient one or more agents that stimulate NK cell function or activity.

13. The method of claim 12, wherein said one or more agents are selected from the group consisting of IL-2, IL-12, IL-15, IL-17, IL-21, IFN-alpha, and IFN-gamma.

14. The method of claim 13, wherein said one or more agent is IL-2.

15. The method of claim 10, further comprising, prior to collecting the biological sample, administering to the patient one or more agents that can (i) stimulate proliferation of stem cells and/or progenitor cells, and/or (ii) stimulate mobilization of stem cells and/or progenitor cells to the peripheral circulation.

16. The method of claim 15, wherein the one or more agents are selected from the group consisting of G-CSF, GM-CSF, SCF, IL-2, IL-7, IL-8, IL-12, and flt-3 ligand.

17. The method of claim 9, further comprising, prior to administering the autologous lymphocytes to the patient, contacting the autologous lymphocytes with one or more agents that stimulate function or activity of NK cells.

18. The method of claim 17, wherein the one or more agents are selected from the group consisting of IL-2, IL-12, IL-15, IL-17, IL-21, IFN-alpha, and IFN-gamma.

19. The method of claim 18, wherein the agent is IL-2.

20. The method of claim 9, further comprising, prior to administering the autologous lymphocytes to the patient, subjecting the patient to an immunosuppressive treatment.

21. The method of claim 20, wherein said immunosuppressive treatment is radiotherapy or chemotherapy.

22. The method of claim 20, wherein said immunosuppressive treatment is surgery with anesthesia.

23-32. (canceled)
